SEQUENCE LISTING (7 sequences)

SEQ ID NO: 1 - DNA, 78 bp, Artificial Sequence
  Synthetic
atgaaaattaaaaccggcgcgcgcattctggcgctgagcgcgctgaccaccatgatgtttagcgctagcgcgctggcc

SEQ ID NO: 2 - DNA, 78 bp, Artificial Sequence
  Synthetic
atgaccattaactttcgccgcaacgcgctgcagctgagcgtggcggcgctgtttagcagcgcgtttatggcgaacgcc

SEQ ID NO: 3 - DNA, 96 bp, Artificial Sequence
  Synthetic
atggatcgccgccgctttattaaaggcagcatggcgatggcggcggtgtgcggcaccagcggcattgctagcctgtttagccaggcggcgtttgcc

SEQ ID NO: 4 - DNA, 126 bp, Artificial Sequence
  Synthetic
atgaacaacaacgatctgtttcaggcgagccgccgccgctttctggcgcagctgggcggcctgaccgtggcgggcatgctggggcccagcctgctgaccccgcgccgcgcgaccgcggcgcaggcc

SEQ ID NO: 5 - DNA, 90 bp, Artificial Sequence
  Synthetic
atgagcggcctgccgctgattagccgccgccgcctgctgaccgcgatggcgctgagcccgctgctgtggcagatgaacaccgcgcatgcc

SEQ ID NO: 6 - DNA, 57 bp, Artificial Sequence
  Synthetic
atgaaaaaaatttggctggcgctggcgggcctggtgctggcgtttagcgctagcgcc

SEQ ID NO: 7 - DNA, 63 bp, Artificial Sequence
  Synthetic
atgaaaaaaaccgcgattgcgattgcggtggcgctggcgggctttgcgaccgtggcgcaggcc

(19) United States
(12) Patent Application Publication
(10) Pub. No.: US 2014/0154742 A1
(43) Pub. Date: Jun. 5, 2014

(54) NOVEL EXPRESSION AND SECRETION VECTOR SYSTEMS FOR HETEROLOGOUS PROTEIN PRODUCTION IN ESCHERICHIA COLI

(71) Applicants: Ayyappan Nair, Mawhaw, NJ (US); Sunil Kumar Sukumaran, Bangalore (IN); Shalaka Samant, Bangalore (IN); Gunja Gupta, Jaipur (IN); Suthakarn Pichaimuthu, Cuddalore (IN); Ganesh Sambasivam, Bangalore (IN)

(72) Inventors: Ayyappan Nair, Mawhaw, NJ (US); Sunil Kumar Sukumaran, Bangalore (IN); Shalaka Samant, Bangalore (IN); Gunja Gupta, Jaipur (IN); Suthakarn Pichaimuthu, Cuddalore (IN); Ganesh Sambasivam, Bangalore (IN)

(73) Assignee: ANTHEM BIOSCIENCES PVT LTD., Karnataka, Bangalore (IN)

(21) Appl. No.: 14/110,320

(22) PCT Filed: Apr. 8, 2012
(86) PCT No.: PCT/IB2012/051730

§ 371 Date: Jan. 14, 2014

(30) Foreign Application Priority Data
Apr. 8, 2011 (IN) 1238/CHE/2011
Dec. 6, 2011 (IN) 4239/CHE/2011

(51) Int. Cl. C12N 15/70

(52) U.S. Cl. CPC C12N 15/70
USPC 435/69.1; 435/320.1; 435/252.33; 435/476; 435/488

(57) ABSTRACT

The present invention relates to a recombinant DNA expression/secretion system in E. coli wherein the said system combines the potential of signal peptide-based translocation of recombinant proteins to the periplasmic space of E. coli with membrane brave defective mutants of E. coli to further aid secretion into the extracellular space. The present invention further relates to the expression system which furthermore includes a helper plasmid to drive the expression of translocons to facilitate improved periplasmic secretion of the over-expressed recombinant protein. In addition, this system also facilitates efficient production of specific proteins of interest in E. coli.

### TECHNICAL FIELD

The present invention relates to a recombinant DNA expression/secretion system in Gram-negative prokaryotes such as Escherichia coli including but not restricted to E. coli BL21 DE3 and E. coli K12. More particularly, the invention relates to a system that combines the potential of signal peptide-based translocation of recombinant proteins to the periplasmic space of E. coli with membrane defective mutants of E. coli to further aid secretion into the extracellular space.

### BACKGROUND ART

Prokaryotes have been widely used for the production of recombinant proteins. Controlled expression of the desired polypeptide or protein is accomplished by coupling the gene encoding the protein through recombinant DNA techniques behind a promoter, the activity of which can be regulated by external factors. This expression construct is carried on a vector, most often a plasmid. Introduction of the plasmid carrying the expression construct into a host bacterium and culturing that organism in the presence of compounds that activate the promoter results in high levels of expression of the desired protein. In this way, large quantities of the desired protein can be produced.

E. coli is the most commonly used prokaryote for protein production. Many different varieties of plasmid vectors have been developed for use in E. coli to build expression systems. The different variations employ several different types of promoters, selectable markers and origins of replication where each of the different configurations imparts a unique property to the expression vector. In the most common arrangement, the expressed protein accumulates in the cytoplasm. While this approach is useful for some proteins, not all proteins can be accumulated in the cytoplasm in an active state. Often, when the desired protein is produced at high levels relative to the host proteins, the protein accumulates as an insoluble particle also known as an inclusion body. Proteins which accumulate as inclusion bodies are difficult to recover in an active form.

Two ways of solving this problem are either to export the target protein to the periplasm between the inner and outer membranes or to facilitate secretion into the extracellular space. There are several potential advantages to having a cloned gene product secreted into the periplasmic space/extracellular medium, including: 1) The protein product can avoid cytoplasmic proteases; 2) normally secreted proteins such as hormones, ligninolytic enzymes, and dextranases may only be able to fold in their active conformation in E. coli if secreted; 3) correct formation of disulfide bonds can be facilitated because the periplasmic space provides a more oxidative environment than the cytoplasm. 4) toxic enzymes such as nucleases or proteases cannot be produced in the cytoplasm due to their potential to exert a toxic effect on the host; and 5) ease of purification.

To target proteins to the periplasm, they are expressed as fusions with signal peptide sequences (e.g. PelB, OmpA, DsbA, TorA and MalE) that follow different secretion pathways (e.g. Sec, Tat and SRP). Other strategies include: a. Modification of signal peptides to enhance translocation. b. Use of heterologous signal sequences in E. coli. c. Co-expression of periplasmic chaperones (e.g. Dsb family proteins) d. Proteasenegative mutant strains to reduce proteolysis.

Export of recombinant proteins to the periplasm of E. coli is in many cases preferable to cytoplasmic production. However, when the protein is overexpressed, export efficiency decreases significantly and some advantages of the system are lost. To avoid overloading the host's translocation machinery following overexpression of signal peptide-recombinant protein fusions attempts have been made to supplement the native secretion machinery with the corresponding translocons from secretory pathways.

Extracellular production of recombinant proteins has several advantages over secretion into the periplasm. Extracellular production does not require outer-membrane disruption to recover target proteins, and, therefore, it avoids intracellular proteolysis by periplasmic proteases and allows continuous production of recombinant proteins.

A number of methods have been applied to promote extracellular secretion of recombinant proteins from E. coli. These include the use of biochemicals, physical methods (osmotic shock, freezing and thawing), lysozyme treatment, and chloroform shock. However, these methods can be applied only after harvesting cells. E. coli normally does not secrete proteins extracellularly except for a few classes of proteins such as toxins and hemolysin. In general, movement of recombinant proteins from the periplasm to the culture medium is the result of compromising the integrity of the outer membrane.

Compromising the integrity of the outer membrane of bacteria can be achieved by a number of approaches. One method involves fusing the product to a carrier protein that is normally secreted into the medium (e.g. hemolysin), or to a protein expressed on the outer membrane (e.g. OmpF).

Proteins secreted into the E. coli periplasm can also be released into the culture medium by co-expression of kil or the gene coding for the third topological domain of the transmembrane protein TolA (TolAIII) or the out genes from E. chrysanthemi EC16. Bacteriocin release protein (BRP) can also be used in the extracellular production of recombinant proteins in E. coli.

Another approach to the extracellular production of target proteins uses L-form cells, wall-less, or wall-deficient cells. Knockouts of outer membrane proteins (omp, tol, lpp, env) have been constructed and the corresponding leaky strains have been used to facilitate secretory recombinant protein expression.

E. coli K12 is a GRAS organism which makes it a safe system for the large scale production of therapeutic proteins. However, K12 is not commonly regarded as a secretory recombinant protein expression strain. An approach towards generation of an efficient secretory E. coli K12 strain as described in this invention combines the potential of signal peptide-protein fusions and over-expression of translocon components with membrane defective mutants.

The present invention utilizes the power of novel signal peptides whose nucleotide sequence are chosen such that they can direct the protein of interest to different cellular compartments of the E. coli cell. Hence, this invention offers a platform of seven different signal peptides that can be tested for determining the best combination possible for secretory expression of the protein of interest.

### SUMMARY OF THE INVENTION

The present invention relates to a recombinant DNA expression/secretion system in Gram-negative prokaryotes such as an Escherichia coli, including but not restricted to E. coli K12 or E. coli BL21 DE3. The said system combines the potential of signal peptide-based translocation of recombinant proteins to the periplasmic space of E. coli with membrane defective mutants to further aid secretion into the extracellular space.

Another aspect of the present invention is an expression vector which optionally includes a helper plasmid which facilitates the expression of translocons to facilitate improved periplasmic secretion of the over-expressed recombinant protein. The system can further be used for production of specific proteins secreted by the E. coli host where normally such proteins are not secreted by the host. In addition, this system also facilitates efficient production of specific proteins of interest in E. coli. The expression vector comprises secretory signal sequence, inducible promoter and a gene of interest.

Yet another aspect of the present invention is a method of obtaining a recombinant cell, said method comprises acts of—(a) obtaining a recombinant vector, (b) transforming a host cell with the recombinant vector and (c) optionally co-transforming the host cell with a helper plasmid to obtain the recombinant cell;

Yet another aspect of the present invention is a method of obtaining a recombinant peptide, said method comprising acts of—(a) obtaining recombinant vector comprising a secretory signal sequence, an inducible promoter and a gene of interest (b) transforming a host cell with the recombinant vector and optionally, co-transforming the host cell with a helper plasmid, (c) expressing the recombinant vector and secreting the recombinant peptide into an extracellular medium and (d) optionally purifying to obtain the recombinant peptide.

Yet another aspect of the present invention is a kit for obtaining recombinant peptide, said kit comprising an expression vector, a recombinant cell or combinations thereof; and a method of assembling a kit for obtaining recombinant peptide, said method comprising act of combining expression vector, recombinant cell or combinations thereof.

### BRIEF DESCRIPTION OF DRAWINGS

In order that the invention be readily understood and put into practical effect, reference will now be made to exemplary embodiments as illustrated with reference to the accompanying figures. The figures together with a detailed description below, are incorporated in and form part of the specification, and serve to further illustrate the embodiments and explain various principles and advantages, in accordance with the present invention.

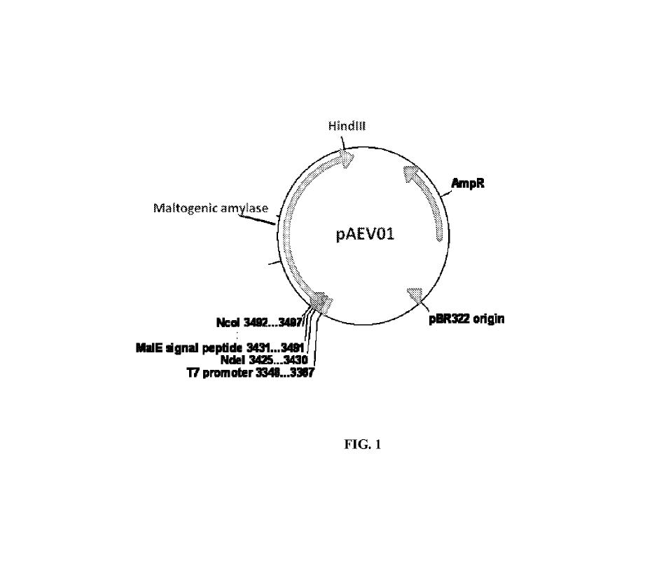

In accordance with the embodiments of the present invention, FIG. 1 depicts the schematic representation of Plasmid map for the expression vector, pAEV01 carrying the signal peptide-target protein fusion under the control of an inducible T5 promoter.

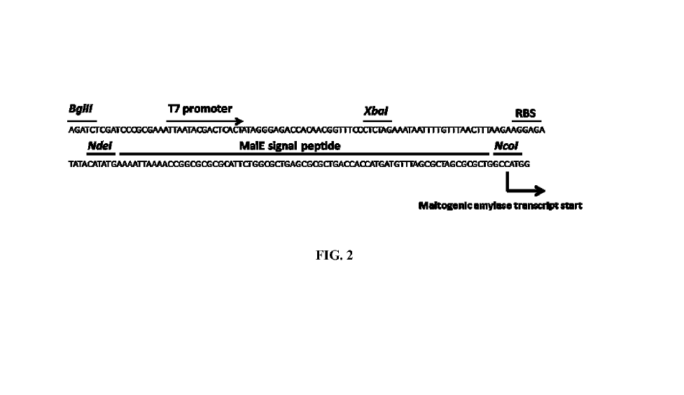

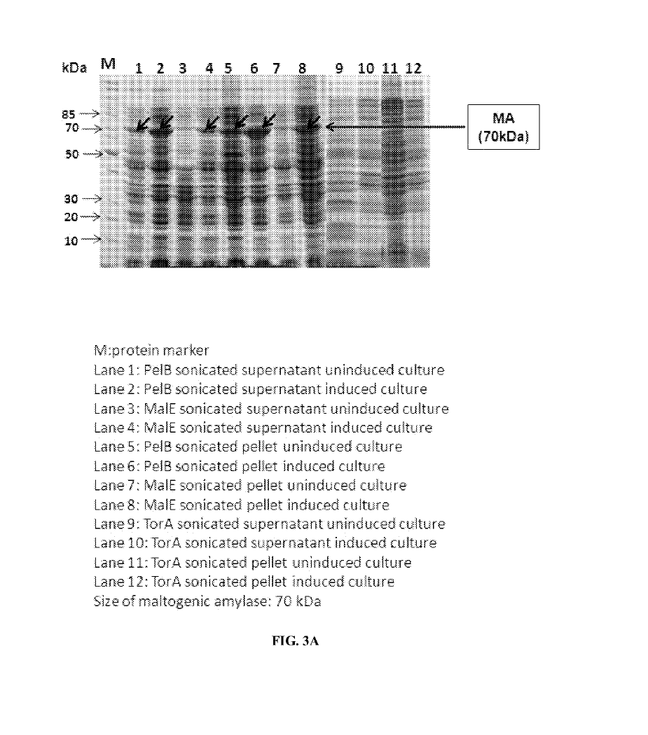

In accordance with the embodiments of the present invention, FIG. 2 is a schematic representation of the T7 promoter, signal peptide sequence, ribosome binding site and transcription start site of Bacillus stearothermophilus maltogenic amylase

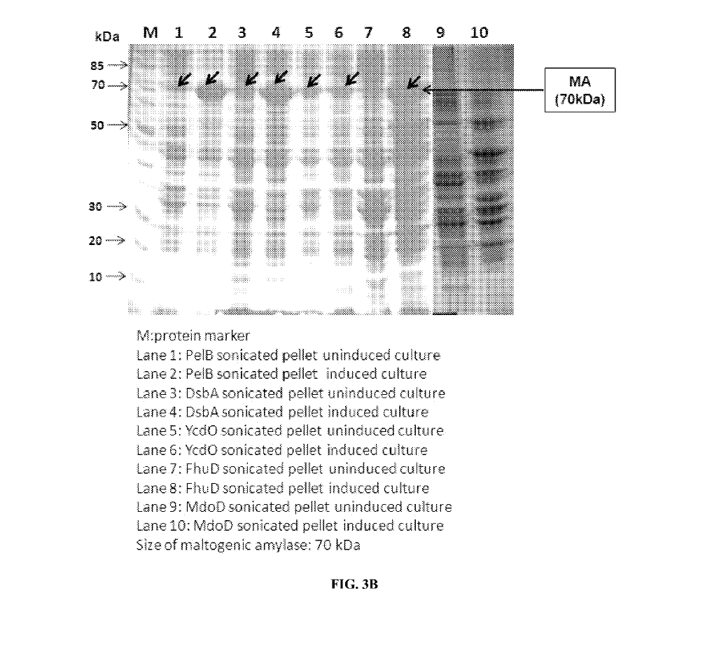

In accordance with the embodiments of the present invention, FIG. 3A shows 0.1 mM IPTG induction followed by SDS-PAGE analysis of lysates from strains expressing MalE, TorA and pelB signal peptide fusions to maltogenic amylase.

In accordance with the embodiments of the present invention, FIG. 3B shows 0.1 mM IPTG induction studies followed by SDS-PAGE analysis of lysates from strains expressing DsbA, YcdO, FhuD, MdoD and pelB signal peptide fusions to maltogenic amylase.

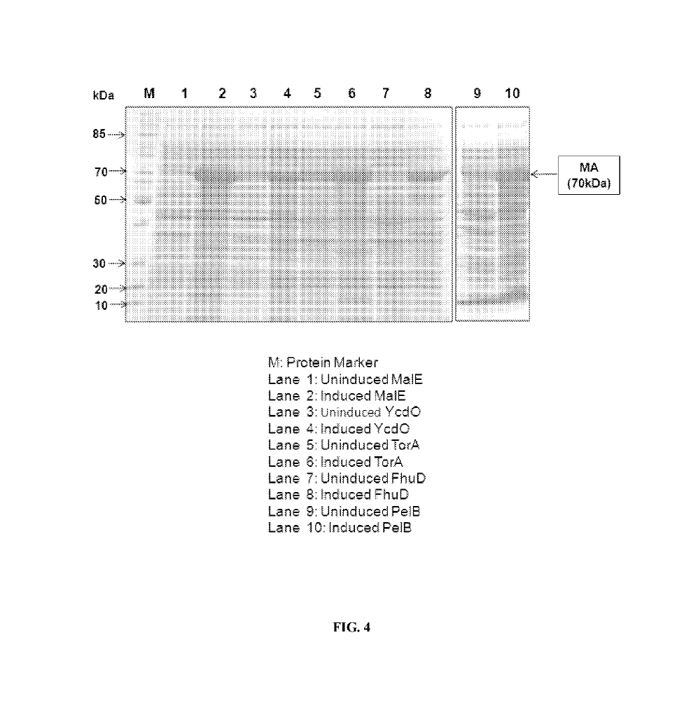

In accordance with the embodiments of the present invention, FIG. 4 shows 1 mM IPTG induction studies followed by SDS-PAGE analysis of lysates from strains expressing MalE, YcdO, TorA, FhuD and pelB signal peptide fusions to maltogenic amylase.

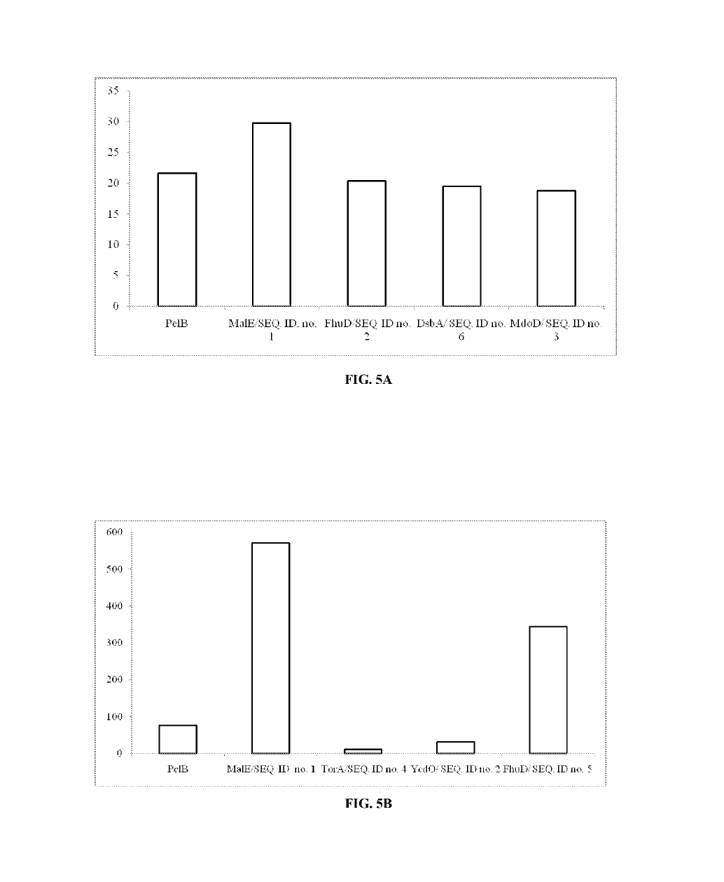

In accordance with the embodiments of the present invention, FIG. 5A shows Fold induction with 0.1 mM of maltogenic amylase activity fused to pelB, MalE, FhuD, DsbA and MdoD compared to corresponding uninduced cultures.

In accordance with the embodiments of the present invention, FIG. 5B shows fold induction with 1 mM of maltogenic amylase activity fused to MalE, FhuD, TorA, YcdO and pelB compared to corresponding uninduced cultures.

### BRIEF DESCRIPTION OF TABLES

In accordance with the embodiments of the present invention, Table 1 lists amino acid residues of the signal sequences and respective export pathways.

In accordance with the embodiments of the present invention, Table 2 lists signal sequences.

In accordance with the embodiments of the present invention, Table 3 lists signal sequence plasmid information.

### DETAILED DESCRIPTION OF THE INVENTION

In order to more clearly and concisely describe and point out the subject matter of the claimed invention, the following definitions are provided for specific terms which are used in the following written description.

By the term ‘Expression’ we mean transcription or translation, or both, as context requires.

By the term an ‘Expression vector’ we refer to recombinant DNA molecule containing the appropriate control nucleotide sequences (e.g., promoters, enhancers, repressors, operator sequences and ribosome binding sites) necessary for the expression of an operably linked nucleotide sequence in a particular host cell. The expression vector may be self-replicating, such as a plasmid, and may therefore carry a replication site, or it may be a vector that integrates into a host chromosome either randomly or at a targeted site. The expression vector may contain a selection gene as a selectable marker for providing phenotypic selection in transformed cells. The expression vector may also contain sequences that are useful for the control of translation.

By the term ‘operably linked/linking’ or ‘in operable combination’ we refer to nucleotide sequence positioned relative to the control nucleotide sequences to initiate, regulate or otherwise direct transcription and/or the synthesis of the desired protein molecule.

By the term ‘Nucleotide’ we refer to a ribonucleotide or a deoxyribonucleotide. ‘Nucleic acid’ refers to a polymer of nucleotides and may be single- or double-stranded. ‘ Polynucleotide’ refers to nucleic acid that is twelve or more nucleotides in length.

By the term ‘Nucleotide sequence of interest’ we mean nucleotide sequence that encodes a ‘protein, polypeptide or peptide sequence of interest,’ the production of which may be deemed desirable for any reason, by one of ordinary skill in the art. Such nucleotide sequences include, but are not limited to, coding sequences of structural genes, regulatory genes, antibody genes, enzyme genes, etc., or portions thereof. The nucleotide sequence of interest may comprise the coding sequence of a gene from one of many different organisms.

A nucleotide sequence ‘encodes’ or ‘codes for’ a protein if the nucleotide sequence can be translated to the amino acid sequence of the protein. The nucleotide sequence may or may not contain an actual translation start codon or termination codon.

A ‘protein, polypeptide or peptide sequence of interest’ is encoded by the ‘nucleotide sequence of interest.’ The protein, polypeptide or peptide may be a protein from any organism, including but not limited to, mammals, insects, micro-organisms such as bacteria and viruses. It may be any type of protein, including but not limited to, a structural protein, a regulatory protein, an antibody, an enzyme, an inhibitor, a transporter, a hormone, a hydrophilic or hydrophobic protein, a monomer or dimer, a therapeutically-relevant protein, an industrially-relevant protein, or portions thereof.

A ‘peptide’ is polymer of four to 20 amino acids, a ‘polypeptide’ is a polymer of 21 to 50 amino acids and a ‘protein’ is a polymer of more than 50 amino acids.

By the term ‘Portion’ when used in reference to a protein we refer to fragments of that protein. The fragments may range in size from four amino acid residues to the entire amino acid sequence of the protein, minus one amino acid.

By the term ‘Purified’ or ‘to purify’ we refer to removal of undesired components from a sample. For example, to purify the secreted protein from growth medium, may mean to remove other components of the medium (i.e., proteins and other organic molecules), thereby increasing the percentage of the secreted protein. The terms ‘ modified’, ‘mutant’ or ‘variant’ are used interchangeably herein, and refer to: (a) a nucleotide sequence in which one or more nucleotides have been added or deleted, or substituted with different nucleotides or modified bases or to (b) a protein, peptide or polypeptide in which one or more amino acids have been added or deleted, or substituted with a different amino acid. A variant may be naturally occurring, or may be created experimentally by one of skilled in the art. A variant may be a protein, peptide, polypeptide or polynucleotide that differs (i.e., an addition, deletion or substitution) in one or more amino acids or nucleotides from the parent sequence.

By the term ‘Periplasm’ we refer to gel-like region between the outer surface of the cytoplasmic membrane and the inner surface of the lipopolysaccharide layer of gram-negative bacteria.

By the term ‘Secretion’ we refer to the excretion of the recombinant protein that is expressed in a bacterium to the periplasm or extracellular growth medium.

In accordance with preferred embodiments, the present invention relates to an expression vector comprising secretory signal sequence, inducible promoter and gene of interest. The present invention further relates to a recombinant cell comprising said vector, optionally alongwith helper plasmid, wherein, said recombinant cell is a membrane defective cell.

Another preferred embodiment of the present invention relates to a method of obtaining recombinant cell, said method comprising steps of: a. obtaining recombinant vector, b. transforming host cell with the recombinant vector; and c. optionally co-transforming the host cell with helper plasmid to obtain the recombinant cell.

Another embodiment relates to a method of obtaining recombinant peptide, said method comprising steps of: a. obtaining recombinant vector comprising secretory signal sequence, inducible promoter and gene of interest; b. transforming host cell with the recombinant vector and optionally, co-transforming the host cell with helper plasmid; c. expressing the recombinant vector and secreting the recombinant peptide into extracellular medium; and d. optionally purifying to obtain the recombinant peptide.

In an embodiment of the present invention, the said secretory signal sequence is codon optimized sequence selected from group comprising SEQ ID NO 1 to SEQ ID NO 7; and the inducible promoter is T5 promoter.

In an embodiment of the present invention, said gene of interest is selected from group comprising prokaryotic and eukaryotic genes.

In yet another embodiment of the present invention, said cell is a prokaryotic cell, preferably an E. coli K12; and the helper plasmid is a plasmid carrying chaperons or translocons from prokaryotic secretory system.

Another embodiment of the present invention relates to a kit for obtaining recombinant peptide, said kit comprising expression vector, recombinant cell or a combination thereof.

The present invention further includes a method of assembling a kit for obtaining recombinant peptide, said method comprising act of combining expression vector, recombinant cell or combinations thereof.

In yet another embodiment of the present invention, the helper plasmid is selected from group comprising of plasmids carrying any component of the chaperones or translocons from the bacterial secretory machinery; illustratively, SEC and TAT.

The present invention furthermore relates to a method for producing a recombinant protein, polypeptide or peptide of interest through secretion of the recombinant protein, polypeptide or peptide to the extracellular growth medium.

In an embodiment of the present invention, the method utilizes expression vectors carrying particular codon optimized variations of native E. coli secretory signal sequences to direct the secretion of the recombinant protein, polypeptide or peptide to the periplasm via the SEC, TAT or SRP export pathways alone or in combination (Table 1).

In an embodiment of the present invention, the expression vector carries a signal sequence selected from the group consisting of SEQ ID NO: 1, SEQ ID NO: 2, SEQ ID NO: 3, SEQ ID NO:4, SEQ ID NO: 5, SEQ ID NO: 6 or SEQ ID NO: 7; downstream of an inducible T5 promoter (Table 2).

In one embodiment, the expression vector is selected from the group consisting of plasmids pAEV01, pAEV02, pAEV03, pAEV04, pAEV05, pAEV06, pAEV07 (Table 3).

In one embodiment, the expression vector is for use in a prokaryotic host cell, for example, Escherichia coli or a strain thereof.

In yet another embodiment of the present invention, an isolated host cell transformed by any of the expression vectors is provided, such that the cell expresses and secretes a protein, polypeptide or peptide of interest encoded by the nucleic acid. In one embodiment, the host cell is a prokaryotic host cell, for example, Escherichia coli or a strain thereof.

In still another embodiment of the present invention, the use of signal peptides corresponding to sequence ID. No. 2, 3 and 5 that harness the features of two periplasmic secretion signals in a single nucleotide sequence is described. Thus the use of the corresponding expression vectors can avoid the clogging of a particular secretion pathway and improve protein yields. The expression vectors described in this invention will carry the signal-peptide target protein fusion under the control of an inducible T5 promoter thus making them amenable to use in an E. coli K12 host for inducing heterologous protein expression.

In still another embodiment of the present invention, the use of helper plasmids to co-express translocons belonging to the SEC and TAT secretory pathways is described. In particular one of these helper plasmids will encode the genes secY, secE and secG as a single operon. Another helper plasmid will encode tatA, tatB and tatC as a single operon. Both these translocon encoding operons will be under the control of an inducible T5 promoter thus making them amenable to use in an E. coli K12 host for inducing heterologous protein expression.

In still another embodiment of the present disclosure, an E. coli strain that has a defective outer membrane co-transformed with the signal peptide-recombinant protein fusion vector and the translocon encoding plasmid. This E. coli strain will not only target the recombinant protein to the periplasmic space but will also facilitate passive diffusion of its leaky protein across the outer membrane into the extracellular medium.

### EXAMPLES

In order that this invention to be more fully understood the following preparative and testing examples are set forth. These examples are for the purpose of illustration only and are not to be construed as limiting the scope of the invention in any way.

### Example 1

### Cloning of Maltogenic Amylase

The following example illustrates the cloning of maltogenic amylase coding gene into pET20b+ and replacement of the pelB signal peptide in pET20b+Maltogenic amylase (MA) with seven other signal peptides described in this invention SEQ ID NO: 1, 2, 3, 4, 5, 6 and 7.

Maltogenic amylase gene from Bacillus stearothennophilus was amplified using primers carrying NcoI and BamHI sites. The primer sequences are: MANcoI fwd primer: 5′-gatcgtaccatgggaATGAGCAGTTCCGCAAGCGT-3′ and MABglIII rev primer: 5′-gatcgtacagatctTCTAGACTAGTTTTGCCACG-3′.

Next, this PCR product was digested with NcoI and BglII enzymes and cloned into the NcoI and BamHI digested pET-20b(+) vector. The resulting ligation mix was transformed into DH5α E. coli cells. The plasmid was sequence verified to ascertain correct maltogenic amylase coding gene and was then digested with NdeI and NcoI to remove pelB signal peptide by gel elution. Seven signal peptides (SEQ. ID. No. 1, 2, 3, 4, 5, 6 and 7) were synthesized with NdeI and NcoI overhangs and cloned into this vector. The resulting vectors retained the reading frame defined by the ATG start codon from pET-20b(+) (FIG. 2). These seven plasmids were transformed into DH5α E. coli cells. Plasmid was isolated from DH5α E. coli cells, validated and then used to transform BL21 DE3 (the producer strain in which heterologous gene expression can be induced using IPTG).

### Example 2

This example illustrates the induction studies on the different maltogenic amylase signal peptide fusions using SDS-PAGE and maltogenic amylase activity assays.

All Eight BL21(DE3) strains were grown in minimal medium supplemented with glucose as the carbon source and 100 ug/ml ampicillin kept overnight in an incubator shaker 37° C., 200 rpm. This culture was diluted 1:100 into a fresh 250 ml flask with 50 ml yeast extract media containing ampicillin and grown at 37° C. in a shaker incubator at 200 rpm. 0.1 mM IPTG was added to the culture when the OD at 600 nm reached 0.6. Culture was then incubated at 26° C. for 16 h at 200 rpm. The induced and uninduced cultures (grown the same way as the induced cultures except no IPTG was added) were pelleted down at 3500 rpm for 15 minutes. The pellet was re-suspended in sample buffer containing 10 mM NaCl, pH 5. This pellet was sonicated to release the soluble protein, cell debris was pelleted out and the supernatant was analyzed on an SDS-PAGE. Similarly induction was carried out by adding 1 mM IPTG to the cultures and induction temperature was maintained at 30° C.

Following induction with 0.1 mM IPTG and grown for 16 h at 26° C. strains carrying pAEV01, pAEV05, pAEV06 and pET-20b(+) construct revealed a significant protein band at 70 kDa, the expected size of maltogenic amylase on a 12% SDS-PAGE. Induction of maltogenic amylase protein levels in pAEV06 and pAEV01 was comparable to that of the parent vector and that of pAEV05 was higher than the parent (FIGS. 3A and 3B). There was no maltogenic amylase produced in strains carrying pAE04 and pAEV07.

pAEV03 plasmid carrying strain showed induction of a truncated protein and the pAEV02 strain showed significant leaky expression of maltogenic amylase i.e. there was no difference in the amount of maltogenic amylase produced with and without induction (FIGS. 3A and 3B).

Experiments to determine the localization of the maltogenic amylase protein will be carried out. This will shed light on the secretory nature of the signal peptide fusions. Our data suggests that fusions to different signal peptides would contribute differently to the expression secretion of different proteins of interest.

Following induction with 1 mM IPTG and grown for 6 h at 30° C. strains carrying pAEV01, pAEV02, pAEV04, pAEV05 and pET-20b(+) construct revealed a significant protein band at 70 kDa, the expected size of maltogenic amylase on a 12% SDS-PAGE (FIG. 4). This data indicates that different IPTG concentrations and temperature post induction play an important role in heterelogous protein expression.

The sonicated supernantant was also subjected to determination of maltogenic amylase activity using the glucose oxidase method. Higher fold induction in terms of maltogenic amylase activity was observed in the pAEV01 construct compared to parent when the cultures were induced with 0.1 mM IPTG (FIG. 5A).

Both the pAEV05 and pAEV01 construct transformed strains showed higher fold induction in terms of maltogenic amylase activity compared to parent when the cultures were induced with 1 mM IPTG (FIG. 5B). Strains carrying other constructs that showed maltogenic amylase expression on an SDS-PAGE exhibited similar functional maltogenic amylase activity as the parent (FIGS. 5A and 5B).

Results indicate that a much higher amount of functional maltogenic amylase is getting targeted into the periplasmic space compared to the parent plasmid in the case of pAEV01 and pAEV05. MalE and FhuD appear to represent improved signal peptides compared to pelB for maltogenic amylase localization to the E. coli periplasmic space.

TABLE 1

| Signal | | Amino acid sequence for | Resulting |
|---|---|---|---|
| sequence | Pathway | each signal sequence | plasmid |
| MalE | SEC | MKIKTGARILALSALTTMMFS | pAEV01 |
| | | ASALApAEV01 | |
| YcdO | TAT + SEC | MTINFRRNALQLSVAALFSSA | pAEV02 |
| | | FMANApAEV02 | |
| MdoD | TAT + SEC | MDRRRFIKGSMAMAAVCGTS | pAEV03 |
| | | GIASLFSQAAFA | |
| TorA | TAT | MNNNDLFQASRRRFLAQLGG | pAEV04 |
| | | LTVAGMLGPSLLTPRRATAA | |
| | | QAA | |
| FhuD | TAT + SEC | MSGLPLISRRRLLTAMALSPL | pAEV05 |
| | | LWQMNTAHA | |
| DsbA | SRP | MKKIWLALAGLVLAFSASAA | pAEV06 |
| | | Q | |
| OmpA | SEC | MKKTAIAIAVALAGFATVAQ | pAEV07 |
| | | A | |

TABLE 2

| | |
|---|---|
| Seq ID no. 1 | atgaaaattaaaaccggcgcgcgcattctggcgctgagcgcgctgaccaccatgatgtttagcg |
| “MalE” | ctagcgcgctggcc |
| Seq ID no. 2 | atggatcgccgccgctttattaaaggcagcatggcgatggcggcggtgtgcggcaccagcggc |
| “YcdO” | attgctagcctgtttagccaggcggcgtttgcc |
| Seq ID no. 3 | atggatcgccgccgctttattaaaggcagcatggcgatggcggcggtgtgcggcaccagcggc |
| “MdoD” | attgctagcctgtttagccaggcggcgtttgcc |
| Seq ID no. 4 | atgaacaacaacgatctgtttcaggcgagccgccgccgctttctggcgcagctgggcggcctga |
| “TorA” | ccgtggcgggcatgctggggcccagcctgctgaccccgcgccgcgcgaccgcggcgcagg |
| | cc |
| Seq ID no. 5 | atgagcggcctgccgctgattagccgccgccgcctgctgaccgcgatggcgctgagcccgctg |
| “FhuD” | ctgtggcagatgaacaccgcgcatgcc |
| Seq ID no. 6 | atgaaaaaaatttggctggcgctggcgggcctggtgctggcgtttagcgctagcgcc |
| “DsbA” | |
| Seq ID no. 7 | atgaaaaaaaccgcgattgcgattgcggtggcgctggcgggctttgcgaccgtggcgcaggcc |
| “OmpA” | |

TABLE 3

| | | Total | |
|---|---|---|---|
| | | base | |
| Plasmid | Secretion | pair | |
| Name | Pathway | (bp) | Signal Sequence |
| pAEV01 | SEC | 5,784 | atgaaaattaaaaccggcgcgcgcattctggcgctgagcgcgctgaccaccat |
| | | | gatgtttagcgctagcgcgctggcc |
| pAEV02 | TAT + SEC | 5,784 | atgaccattaactttcgccgcaacgcgctgcagctgagcgtggcggcgctgttt |
| | | | agcagcgcgtttatggcgaacgcc |
| pAEV03 | TAT + SEC | 5,802 | atggatcgccgccgctttattaaaggcagcatggcgatggcggcggtgtgcgg |
| | | | caccagcggcattgctagcctgtttagccaggcggcgtttgcc |
| pAEV04 | TAT | 5,832 | atgaacaacaacgatctgtttcaggcgagccgccgccgctttctggcgcagct |
| | | | gggcggcctgaccgtggcgggcatgctggggcccagcctgctgaccccgcg |
| | | | ccgcgcgaccgcggcgcaggcc |
| pAEV05 | TAT + SEC | 5,796 | atgagcggcctgccgctgattagccgccgccgcctgctgaccgcgatggcgc |
| | | | tgagcccgctgctgtggcagatgaacaccgcgcatgcc |
| pAEV06 | SRP | 5,763 | atgaaaaaaatttggctggcgctggcgggcctggtgctggcgtttagcgctag |
| | | | cgcc |
| pAEV07 | SEC | 5,769 | atgaaaaaaaccgcgattgcgattgcggtggcgctggcgggctttgcgaccgt |
| | | | ggcgcaggcc |

Thus, the present invention utilizes the power of novel signal peptides whose nucleotide sequence has been optimized to support efficient translation. These sequences are chosen such that they can direct the protein of interest to different cellular compartments of the E. coli cell. Hence, this invention offers a platform of seven different signal peptides that can be tested for determining the best combination possible for secretory expression of the protein of interest.

SEQUENCE LISTING

GENERAL INFORMATION:
NUMBER OF SEQUENCES: 7
INFORMATION FOR SEQ ID NO: 1:
SEQUENCE CHARACTERISTICS:
LENGTH: 78 BASE PAIRS
TYPE: NUCLEIC ACID
MOLECULE TYPE: DNA
SEQUENCE DESCRIPTION: SEQ ID NO: 1:
atgaaaatta aaaccggcgc gcgcattctg gcgctgagcg cgctgaccac catgatgttt 60
agcgctagcg cgctggcc 78
INFORMATION FOR SEQ ID NO: 2:
SEQUENCE CHARACTERISTICS:
LENGTH: 78 BASE PAIRS
TYPE: NUCLEIC ACID
MOLECULE TYPE: DNA
SEQUENCE DESCRIPTION: SEQ ID NO: 2:
atgaccatta actttcgccg caacgcgctg cagctgagcg tggcggcgct gtttagcagc 60
gcgtttatgg cgaacgcc 78
INFORMATION FOR SEQ ID NO: 3:
SEQUENCE CHARACTERISTICS:
LENGTH: 96 BASE PAIRS
TYPE: NUCLEIC ACID
MOLECULE TYPE: DNA
SEQUENCE DESCRIPTION: SEQ ID NO: 3:
atggatcgcc gccgctttat taaaggcagc atggcgatgg cggcggtgtg cggcaccagc 60
ggcattgcta gcctgtttag ccaggcggcg tttgcc 96
INFORMATION FOR SEQ ID NO: 4:
SEQUENCE CHARACTERISTICS:
LENGTH: 126 BASE PAIRS
TYPE: NUCLEIC ACID
MOLECULE TYPE: DNA
SEQUENCE DESCRIPTION: SEQ ID NO: 4:
atgaacaaca acgatctgtt tcaggcgagc cgccgccgct ttctggcgca gctgggcggc 60
ctgaccgtgg cgggcatgct ggggcccagc ctgctgaccc cgcgccgcgc gaccgcggcg 120
caggcc 126
INFORMATION FOR SEQ ID NO: 5:
SEQUENCE CHARACTERISTICS:
LENGTH: 90 BASE PAIRS
TYPE: NUCLEIC ACID
MOLECULE TYPE: DNA
SEQUENCE DESCRIPTION: SEQ ID NO: 5:
atgagcggcc tgccgctgat tagccgccgc cgcctgctga ccgcgatggc gctgagcccg 60
ctgctgtggc agatgaacac cgcgcatgcc 90
INFORMATION FOR SEQ ID NO: 6:
SEQUENCE CHARACTERISTICS:
LENGTH: 57 BASE PAIRS
TYPE: NUCLEIC ACID
MOLECULE TYPE: DNA
SEQUENCE DESCRIPTION: SEQ ID NO: 6:
atgaaaaaaa tttggctggc gctggcgggc ctggtgctgg cgtttagcgc tagcgcc 57
INFORMATION FOR SEQ ID NO: 7:
SEQUENCE CHARACTERISTICS:
LENGTH: 63 BASE PAIRS
TYPE: NUCLEIC ACID
MOLECULE TYPE: DNA
SEQUENCE DESCRIPTION: SEQ ID NO: 7:
atgaaaaaaa ccgcgattgc gattgcggtg gcgctggcgg gctttgcgac cgtggcgcag 60
gcc 63

1. A recombinant host cell comprising an expression vector, optionally along with a helper plasmid, wherein, the said recombinant cell is a membrane defective cell.

2. The expression vector as mentioned in claim 1 is capable of directing the expression and secretion of a protein, polypeptide or a peptide in a suitable host cell, wherein the expression vector further comprises a secretory signal sequence, inducible promoter and a gene of interest.

3. The helper plasmid as mentioned in claim 1 co-express translocons belonging to the SEC, TAT, SRP export pathway or in a combination thereof.

4. The expression vector as mentioned in claim 2 wherein gene of interest comprises a prokaryotic and eukaryotic gene.

5. The expression vector as mentioned in claim 2, wherein the secretory signal sequence is selected from the group consisting of SEQ ID NO:1, SEQ ID NO:2, SEQ ID NO:3, SEQ ID NO: 4, SEQ ID NO: 5, SEQ ID NO: 6, SEQ ID NO: 7, SEQ ID NO: 8 or SEQ ID NO: 9.

6. The expression vector as mentioned in claim 2, is used in a host cell wherein the host cell is a prokaryote.

7. The isolated host cell of claim 6, wherein said host cell is an Escherichia coli or a strain thereof.

8. The expression vector as mentioned in claim 2 comprises at least one of the plasmids selected from the group consisting of pAEV01, pAEV02, pAEV03, pAEV04, pAEV05, pAEV06 or pAEV07.

9. A method of obtaining a recombinant cell, said method comprising steps of:
a) obtaining a recombinant vector;
b) transforming host cell with the recombinant vector; and
c) optionally co-transforming the host cell with helper plasmid to obtain the recombinant cell.

10. A method of obtaining recombinant peptide, method comprising steps of:
a) obtaining recombinant vector comprising secretory signal sequence, inducible promoter and gene of interest;
b) transforming host cell with the recombinant vector and optionally, co-transforming the host cell with helper plasmid;
c) expressing the recombinant vector and secreting the recombinant peptide into extracellular medium; and
d) optionally purifying the recombinant peptide.

11. The helper plasmid as mentioned in claims 9 and 10 co-express translocons belonging to the SEC, TAT, SRP export pathway or in a combination thereof.

12. The method of claims 9 and 10 wherein expression vector comprises at least one of the plasmids selected from the group consisting of pAEV01, pAEV02, pAEV03, pAEV04, pAEV05, pAEV06 or pAEV07.

13. The method as mentioned in claims 9 and 10, wherein the expression vector comprises a codon optimized secretory signal sequence selected from the group consisting of SEQ ID NO:1, SEQ ID NO:2 SEQ ID NO:3, SEQ ID NO: 4, SEQ ID NO: 5, SEQ ID NO: 6, SEQ ID NO: 7, SEQ ID NO: 8 or SEQ ID NO: 9.

14. The method as mentioned in claim 10 wherein the said inducible promoter is a T5 promoter.

15. The host cell as mentioned in claim 7 is having a defective outer membrane which is further co-transformed with the signal peptide-recombinant protein fusion vector and the translocon encoding plasmid.

16. A kit for obtaining a recombinant peptide, said kit comprising an expression vector, a recombinant cell or a combination thereof.
